(19) United States
(12) Patent Application Publication
(10) Pub. No.: US 2019/0274664 A1
(43) Pub. Date: Sep. 12, 2019

(54) BIOLOGICAL SAMPLE COLLECTION METHOD AND BIOLOGICAL SAMPLE COLLECTION TOOL

(71) Applicant: Shimadzu Corporation, Kyoto (JP)

(72) Inventor: Shinichi IWAMOTO, Kyoto (JP)

(73) Assignee: Shimadzu Corporation, Kyoto (JP)

(21) Appl. No.: 16/298,091

(22) Filed: Mar. 11, 2019

(30) Foreign Application Priority Data
Mar. 12, 2018 (JP) 2018-043974

(51) Int. Cl. A61B 10/02; G01N 33/68

(52) U.S. Cl. CPC A61B 10/02; A61B 2010/0216; G01N 33/6848; G01N 33/6881

(57) ABSTRACT

A test subject takes a component collection tool with his/her fingers, and rolls the tool between pads of the fingers several times to transfer components attached on a skin surface to a surface of the tool. The component collection tool is immersed in a predetermined solvent to allow the components on the skin surface to be dissolved in the solvent. Bligh-Dyer method may be used therefor, for example. From a liquid in which lipid or others are thus collected, a sample for analysis using MALDI-MS or LDI-MS is prepared and subjected to the analysis. Accordingly, components on the skin surface of the test subject can be efficiently collected and subjected to analysis with burden on the test subject being reduced.

### FIELD

The present invention relates to a method for collecting biological samples and a tool for the use of collecting biological samples. In detail, the present invention relates to a biological sample collection method for collecting components existing on skin surfaces of human fingers and palms, and a tool for use in the method, for subjecting the components to various analyses.

### BACKGROUND

On the skin surfaces of human fingers, fatty acids, cholesterol, neutral fat, and such various components (compounds) that ooze out from the body of a test subject are adhered. Such components are important for recognizing the health condition of a test subject, determining the severity level of a disease, or verifying the effects of medical treatment on the disease. In a typical health examination, blood collected from a test subject is analyzed for verifying the content or other factors relating to such components derived from a living body. Here, if components on the surfaces of test subject's fingers can be used for performing such examinations accurately, the physical load on the test subject can be reduced, and accordingly, the health examination can be performed at a high rate of frequency.

As the method for analyzing components existing on surfaces of test subject's fingers, a method for performing mass spectrometry after collecting samples has been known, as described below.

Specifically, a fingertip of a test subject is first pressed against a surface of a sample plate dedicated to the method, so that components on surfaces of the test subject's fingers are transferred to the surface of the plate. The components transferred to the plate are subjected to mass spectrometry, using a matrix-assisted laser desorption/ionization mass spectrometer (MALDI-MS) or a laser desorption/ionization mass spectrometer (LDI-MS) which does not use matrix, so as to collect data including mass spectra. The data is analyzed, to identify the components or to estimate the content of the components.

Non Patent Literature 1 discloses a plate that is prepared with processing on iron-oxide nanoparticles and is produced for use in a surface assisted laser desorption/ionization (SALDI) method, and is additionally used as the sample plate to which components on the skin surface of a fingertip are transferred. Non Patent Literature 1 also reports that the components on the skin surface were transferred without using such a special sample plate, but with using a standard sample plate made of stainless steel, which is used in MALDI method and other methods, and analysis was carried out on the thus obtained components.

Analysis using, as another method, a sample collection method that is known as tape stripping has also been known. In this method, an adhesive tape is adhered to a fingertip of a test subject, and then peeled off from the fingertip, to thereby collect stratum corneum of the fingertip on the tape. Then, the adhesive tape is immersed in an appropriate solvent, to allow components contained in the stratum corneum adhered to the tape to be dissolved and collected, thereby preparing a sample for mass spectrometry. According to this method, the stratum corneum at the fingertip of the test subject can be collected relatively easily and accurately, and thus is subjected to analysis.

Non Patent Literature 1: “Laser Desorption/Ionization Mass Spectrometry (LDI-MS) of Lipids with Iron Oxide Nanoparticle-Coated Targets”, Mass Spectrometry, Vol. 3, No. 1, 2014, A0026

### SUMMARY

There are, unfortunately, problems illustrated hereinafter regarding the aforementioned conventional methods for collecting components on skin surfaces.

Typically, a certain amount of components is required in mass spectrometry, for obtaining signals derived from a component at an adequately high intensity. Accordingly, a test subject is required to strongly press his/her fingertip against a sample plate several times in a method in which components on the skin surface are transferred to a sample plate, as described earlier. Although sample collection is noninvasive, it puts a large burden on a test subject, particularly when the test subject is an aged person or is a patient afflicted with a disease. In the first place, in MALDI-MS and LDI-MS, an area that can be irradiated with a laser beam at once is remarkably smaller (a level of 0.1 mm in diameter) than an area where components are transferred (usually, a level of 20 to 30 mm in diameter). Accordingly, the amount of components ionized at once is merely a level of one several-ten-thousandths of components collected on the sample plate. Thus, efficiency in sample usage is low. For increasing the efficiency in sample usage, it is necessary for the analysis to be repeated several times by moving the position to be irradiated with a laser beam, and to integrate signals. Such analysis takes time and has difficulty in increasing the throughput. In addition, though a sample existing on the sample plate can be analyzed using MALDI-MS or LDI-MS, it is difficult to analyze a sample on a sample plate with other methods.

Meanwhile, in the tape stripping method, the efficiency in sample usage is high. However, in this method, a stratum corneum layer that is a barrier layer of skin is peeled off. This imposes a physical burden on a test subject, though the burden is actually slight. In view of this, it is not proper to repeatedly collect samples from an aged person or a patient afflicted with a disease.

The present invention has been made in view of the previous problems. The purpose of the present invention is to provide a biological sample collection method and a tool therefor, which are capable of efficiently collecting components on skin in an amount required for analysis, with preventing the imposition of a physical burden on a test subject as much as possible.

The present invention developed for solving the previously described problem is a biological sample collection method for collecting, as a sample to be subjected to analysis, a component existing on a skin surface of a fingertip, a finger pad, or a palm of a test subject, the method including:

a) transferring the component on the skin surface, using a component collection tool, to a surface of the component collection tool by rolling the component collection tool at the fingertip, the finger pad, or the palm of the test subject, the component collection tool being sized to be holdable by a fingertip, a finger pad, or a palm of a person, made of a material, at least on the surface thereof, being smooth and chemically resistant, and having a surface from which a component adhered to the surface is easily removable; and

b) immersing the component collection tool to which the component on the skin surface is transferred, in a predetermined solvent, to dissolve the component adhered to the surface of the component collection tool into the solvent, thereby to collect the component;

Furthermore, the present invention developed for solving the previously described problem is a biological sample collection tool for collecting, as a sample to be subjected to analysis, a component existing on a skin surface of a fingertip, a finger pad, or a palm of a test subject,

the biological sample collection tool being sized to be holdable by a fingertip, a finger pad, or a palm of a person, made of a material, at least on the surface thereof, being smooth and chemically resistant and having a surface from which components adhered to the surface is easily removable; and

the biological sample collection tool being rolled at the fingertip, the finger pad, or the palm of the test subject, to allow the component on the skin surface to be transferred to the surface of the biological sample collection tool.

In the biological sample collection method according to the present invention, a three-dimensional component collection tool (it is also referred to as the biological sample collection tool, according to the aforementioned invention) is used in place of a flat sample plate that has conventionally been used for performing the transfer of components existing on a skin surface of a finger or a palm of a test subject. This biological sample collection tool: is sized to be holdable by a fingertip, a finger pad, or a palm of a person; is made of a material smooth and chemically resistant at least on its surface; and has a surface from which the components adhered to the surface is easily removable.

The component collection tool has such a size, and the surface thereof is smooth. Accordingly, a test subject, for example, can hold the component collection tool with his/her fingers and roll it easily and smoothly between the pads of the fingers. Alternatively, the test subject can also roll the component collection tool with his/her finger or palm in a state that the component collection tool is placed on a clean surface. If it is difficult for a test subject himself/herself to conduct such an action, it is also easy that a laboratory technician or such a person in charge moves the test subject's hand, or the like, to roll the component collection tool with the component collection tool being in contact with the palms or other parts of the test subject. Accordingly, components on a skin surface of a finger or a palm can be transferred to a wide range of surfaces of the component collection tool. The surface of the component collection tool is smooth. Accordingly, stratum corneum of fingers is hardly peeled off when the tool is rolled between the fingers of the test subject. This reduces the burden on the test subject's body.

Subsequently, the component collection tool to which the component on the skin surface of the test subject is transferred, is immersed in a predetermined solvent, to dissolve the component adhered to the surface of the tool into the solvent. A solvent as appropriate can be used. Typically, water, methanol, chloroform, or other organic solvents may be used. The component collection tool has the chemical resistance at least on its surface, thereby avoiding material itself of the component collection tool from being dissolved. The component collection tool also has a surface from which the components adhered to its surface is easily removable. Thus, when the tool is immersed in the solvent, the components on the skin surface are rapidly dissolved in the solvent. The components on the skin surface hardly remain on the surface of the component collection tool. Accordingly, approximately all components on the skin surface adhered to the surface of the tool can be dissolved in the solvent.

After the component on the skin surface is dissolved in the solvent, the solvent liquid may be subjected to analysis as it is, or a solid sample may be prepared by performing processing such as drying and solidifying on the solvent liquid. Forms of the sample are different depending on analysis methods. When mass spectrometry is performed using LDI-MS, for example, a sample can be prepared by dropping, on a sample plate, the liquid in which components on a skin surface are dissolved, and then by drying and solidifying the liquid.

In the biological sample collection method according to the present invention, a shape of the component collection tool is not particularly limited. Here, the component collection tool may have a circular shape or an elliptical shape at at least a part of its cross section. With this, a test subject can smoothly roll the component collection tool with his/her fingers.

For a specific shape of the component collection tool, one of a spherical shape, spindle shape, and a columnar shape may be adopted.

With these shapes, the fingers of a test subject touch a wide range of the surface of the component collection tool, thereby transferring a large amount of components on the skin surface to a surface of the component collection tool. This increases the amount of the components to be analyzed, and thus improves the sensitivity and accuracy of the analysis.

In the aforementioned configuration, the diameter of the circular shaped cross section or the long diameter of the elliptical shaped cross section of the component collection tool may be in the range of 5 to 20 mm with this, a test subject can roll, without any effort, the component collection tool with his/her fingers or palms. More preferably, the diameter of the circular shaped cross section or the long diameter of the elliptical shaped cross section of the component collection tool may be in the range of 8 to 15 mm with this, a test subject can easily hold the tool by himself/herself with his/her fingers, thereby allowing the tool to remain stable.

It is preferable in the biological sample collection method according to the present invention, that a material for at least the surface of the component collection tool is fluororesin. Typically, polytetrafluoroethylene may be used. Accordingly, an inexpensive component collection tool suitable for collecting components on a skin surface can be provided.

According to the biological sample collection method and the biological sample collection tool relating to the present invention, components on a skin surface in an amount required for analysis can be efficiently collected by preventing a physical burden from being imposed on a test subject. With the present invention, even when a test subject is an elderly person or a patient afflicted by a disease, components on a skin surface can be collected at high frequency. Furthermore, an amount of components to be subjected to the analysis increases, to improve the analysis accuracy and the analysis sensitivity, and to reduce the time period for analysis.

### BRIEF DESCRIPTION OF DRAWINGS

FIG. 1 is an explanatory diagram of a way of collecting components on a skin surface in an embodiment of a biological sample collection method according to the present invention.

FIG. 2 is a flowchart of steps in analysis on components on a skin surface, the steps including the biological sample collection method according to the embodiment.

FIG. 3A is an explanatory diagram of examples of a component collection tool to be used in the biological sample collection method according to the present invention.

FIG. 3B is an explanatory diagram of examples of a component collection tool to be used in the biological sample collection method according to the present invention.

FIG. 3C is an explanatory diagram of examples of a component collection tool to be used in the biological sample collection method according to the present invention.

### DETAILED DESCRIPTION

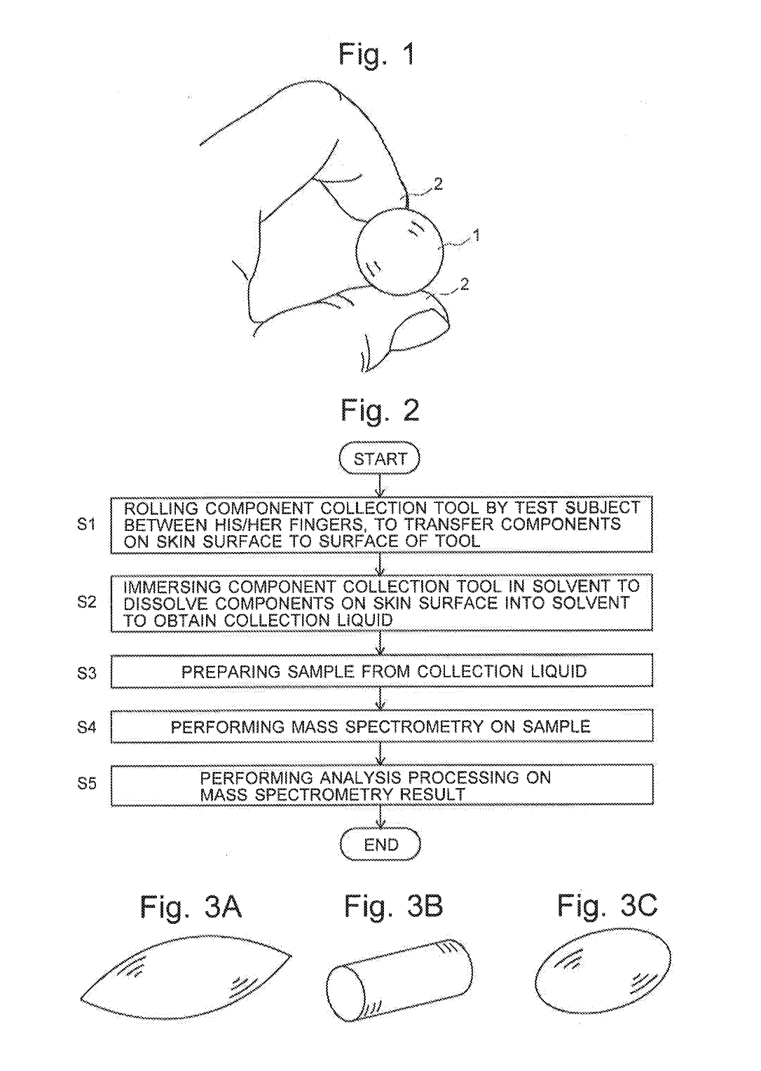

The biological sample collection method according to an embodiment of the present invention is described as follows, with reference to the drawings. FIG. 2 is a flowchart of steps in analysis on components on a skin surface, which include the biological sample collection method according to the present embodiment. FIG. 1 is an explanatory diagram of a way of collecting components on a skin surface in the biological sample collection method according to the present embodiment.

In the biological sample collection method according to the present embodiment, a spherical-shaped component collection tool 1 as shown in FIG. 1 is used for collecting components on a skin surface of a test subject. The component collection tool 1 is made of fluororesin including polytetrafluoroethylene (Teflon: trademark registration) and others, and has a diameter at a level of 10 mm. As the fluororesin, tetrafluoroethylene-perfluoroalkylvinylether copolymer (PFA), tetrafluoroethylene-hexafluoropropylene copolymer (FEP), tetrafluoroethylene-ethylene copolymer (ETFE), polyvinylidene fluoride (PVDF), polychloro-trifluoroethylene (PCTFE), and others are typically used in addition to polytetrafluoroethylene (PTFE).

A material for the component collection tool 1 is not limited, at least on the surface, to the fluororesin. Here, various solvents are used for collecting components on skin surfaces from the component collection tool 1. Accordingly, the material preferably has chemical resistance for preventing being dissolved and/or corroded by such solvents. Furthermore, it is also preferable that the material has heat resistance at a certain level, for possible application of heat upon collection of the components. Furthermore, a material that allows components on the skin surface, such as sebum adhered to the skin surface, to permeate through a surface of the tool, which is made of the material, into the inside thereof is apparently unfavorable. Another favorable requirement is that the material has a surface from which components adhered to the skin surface is easily removable. If a surface of the component collection tool 1 is not smooth, it is difficult for the component collection tool 1 to be rolled by being held by fingers of the test subject. Thus, it is also required for the component collection tool 1 to have a surface that is smooth at an appropriate level. As a material that satisfies such various conditions, fluororesin is favorable. In particular, polytetrafluoroethylene is especially favorable.

The test subject takes, with his/her fingers 2, the component collection tool 1 the surface of which is sufficiently washed, as shown in FIG. 1. Then, the test subject rolls the component collection tool 1 several times between the pads of the fingers 2, to allow components on the skin surface to be transferred to a surface of the component collection tool 1 (Step S1). Typically, even if the test subject rolls the component collection tool 1 with little focus or awareness, fingertips come into contact with various parts on the surface of the component collection tool 1. Accordingly, to the surface of the component collection tool 1, components on the skin surface are approximately uniformly adhered.

An analysis operator then puts the component collection tool 1 to which components have been transferred, into a test tube that accommodates a mixed solution of chloroform, methanol, and water. Thus, water soluble components, protein, and lipid, are separated and collected using the well-known Bligh-Dyer method (Step S2). Specifically, the component collection tool 1 is immersed into the mixed solution containing chloroform, methanol and water respectively at a certain ratio, and the solution is stirred. Accordingly, the components on the skin surface which are adhered to the surface of the component collection tool 1 are dissolved in the mixed solution, and thus a suspension is obtained. Then, the suspension is centrifugalized. After the centrifugation, the suspension is separated into two layers. Most of the lipid molecules are collected in an organic solvent layer that contains chloroform and methanol, and is positioned in the lower side of the two layers. It should be noted that the material of the component collection tool 1 has chemical resistance as mentioned earlier, and thus has high resistance to chloroform. Accordingly, corrosion does not occur on the component collection tool 1 itself.

As previously mentioned, components on the skin surface are approximately uniformly adhered to the entire surface of the component collection tool 1, in general. Accordingly, a sufficiently large amount of components on the skin surface can be collected in comparison with a case where a fingertip is pressed on a sample plate or the like to transfer components on the skin surface thereto.

The collection liquid thus obtained and containing the lipid is concentrated by drying under reduced pressure, and a sample to be subjected to mass spectrometry is then prepared (Step S3). Methods for preparing samples depend on an ionization method to be used in a mass spectrometer. When MALDI method is used, for example, a sample can be prepared by dropping, on a sample plate, concentrated collection liquid to which matrix is previously added, and then drying and solidifying the collection liquid. When LDI method that does not use matrix is used, a sample can be prepared by dropping concentrated collection liquid on a sample plate, and then drying and solidifying the collection liquid.

The sample thus prepared is set to a mass spectrometer, and mass spectrometry is carried out under predetermined conditions (Step S4). The mass spectrometer used for such analysis typically includes a time-of-flight (TOF) mass spectrometer, or TOF/TOF mass spectrometer and an ion trap time-of-flight mass spectrometer which can perform mass spectrometry by allowing ions to be dissociated once or more. If a quadrupole time-of-flight (Q-TOF) mass spectrometer or other mass spectrometers in which an ion source using atmospheric-pressure ionization method, such as an electrospray ionization (ESI) method, is equipped, are used, it is not necessary to prepare a solid sample as mentioned before. In such a case, concentrated collection liquid can be introduced as it is, so as to be subjected to mass spectrometry. Moreover, samples thus prepared can also be applied to analysis using GC-MS, or LC-MS in which a mass spectrometer is combined with liquid chromatograph or gas chromatograph.

Then, a predetermined analysis processing is carried out based on the result of the mass spectrometry, which is, in particular, mass spectrum, obtained by the aforementioned mass spectrometry, thereby identifying the components on a skin surface of the test subject, or determining the quantity thereof (Step S5). As just mentioned, an amount of components collected in Step S2 is sufficiently greater than that obtained by conventional methods. Accordingly, an amount of components contained in the prepared sample is also great. With this, mass spectra can be obtained in which each of the components is detected with high sensitivity. Thus, accuracy in the identification and determination of the quantity is also enhanced. Furthermore, very small quantities of components can also be identified.

FIG. 3 shows examples of a component collection tool having a shape that is not of a spherical shape. FIG. 3(a) shows an example having a spindle shape; FIG. 3(b) shows an example having a columnar shape; and FIG. 3(c) shows an example having a spheroidal shape. Alternatively, a long and slender stir-bar shape may be used. In any case, the component collection tool is only required to have a shape that is easily taken by a test subject with his/her fingers and is easily rolled. It is also preferable for the component collection tool to have a shape allowing test subject's fingers to touch it with a range as large as possible, upon being taken. Accordingly, it is preferable that the component collection tool has, at at least a part of cross section thereof, a shape having a circular shape or an elliptical shape.

Although analysis using the mass spectrometer is performed in the before-mentioned description, the collection liquid obtained through Steps S1 and S2 mentioned before can also be used as a sample for analysis by an approach other than mass spectrometry.

In addition, a solvent to be used for processing in Step S2 is not limited to the aforementioned solvent. An appropriate organic solvent can be used. In other words, a method for collecting lipid is not limited to the Bligh-Dyer method. Here, lipid normally has high composition in the components on a skin surface. Accordingly, collection using chloroform and/or methanol, which is particularly effective in collecting lipid, is preferable.

Although the spherical-shaped component collection tool 1 used in the aforementioned embodiment has the diameter of about 10 mm, the size of the component collection tool 1 is not limited thereto. If components on a skin surface are collected by a test subject himself/herself taking the component collection tool 1 between fingers and rolling it, as mentioned previously, it is preferable that the diameter of the tool 1 is a level of 8 to 15 mm.

Meanwhile, it is often difficult for some test subjects to hold something between their fingers. In such cases, the component collection tool 1 is placed on a clean surface, and the test subjects may roll the tool 1 with their fingers or palms. Thus, components on skin surfaces can be transferred to a surface of the tool 1. An elderly person or others may not move his/her hand by himself/herself. In such cases, the analysis operator or an assistant may hold and move the test subject's hand to allow the component collection tool 1 to be rolled by being touched to the palms or other parts of the test subject. In such a case, the component collection tool 1 may have a larger or smaller size, i.e., the diameter in a range from 5 to 20 mm.

It is apparent that any modification, correction, or addition along the scope of the present invention is included in the scope of claims of the present application, for aspects that are not described earlier.

For example, the sample collection method according to the present invention is intended to collect components on a skin surface of a finger of a person. Here, it may be used in alternative ways.

For example, in order to collect components on skin surfaces of animals kept as pets or farm animals, or experimental animals including rats, the foregoing component collection tool may be rubbed against skin behind the ears of animals, for example, or a part at which components on a skin surface can easily be collected, thereby allowing the components on the skin surface to be transferred to a surface of the component collection tool. Since the component collection tool has a convex-curved surface, the surface is easy to be in contact with skin behind the ears, and other parts, thereby facilitates collecting components on skin surfaces.

1. A biological sample collection method for collecting, as a sample to be subjected to analysis, a component existing on a skin surface of a fingertip, a finger pad, or a palm of a test subject, the method comprising:
a) transferring the component on the skin surface, using a component collection tool, to a surface of the component collection tool by rolling the component collection tool at the fingertip, the finger pad, or the palm of the test subject, the component collection tool being sized to be holdable by a fingertip, a finger pad, or a palm of a person, made of a material, at least on a surface thereof, being smooth and chemically resistant, and having a surface from which a component adhered to the surface is easily removable; and
b) immersing the component collection tool to which the component on the skin surface is transferred, in a predetermined solvent, to dissolve the component adhered to the surface of the component collection tool into the solvent, thereby to collect the component.

2. The biological sample collection method according to claim 1,
wherein the component collection tool has, at at least a part of a cross section thereof, a circular shape or an elliptical shape.

3. The biological sample collection method according to claim 2,
wherein the circular shape or the elliptical shape at the cross section of the component collection tool has a diameter in a range from 5 to 20 mm.

4. The biological sample collection method according to claim 2,
wherein the component collection tool has a spherical shape.

5. The biological sample collection method according to claim 2,
wherein the component collection tool has a spindle shape.

6. The biological sample collection method according to claim 2,
wherein the component collection tool has a columnar shape.

7. The biological sample collection method according to claim 1,
wherein at least the surface of the component collection tool is made of a material that includes fluororesin.

8. A biological sample collection tool for collecting, as a sample to be subjected to analysis, a component existing on a skin surface of a fingertip, a finger pad, or a palm of a test subject,
the biological sample collection tool being sized to be holdable by a fingertip, a finger pad, or a palm of a person, made of a material, at least on a surface thereof, being smooth and chemically resistant and having a surface from which components adhered to the surface is easily removable; and
the biological sample collection tool being rolled at the fingertip, the finger pad, or the palm of the test subject, to allow the component on the skin surface to be transferred to the surface of the biological sample collection tool.

9. The biological sample collection method according to claim 2,
wherein the surface of the component collection tool is made of a material that includes fluororesin.

10. The biological sample collection tool according to claim 8, having, at at a least a part of a cross section thereof, a circular shape or an elliptical shape.

11. The biological sample collection tool according to claim 10,
wherein the circular shape or the elliptical shape at the cross section of the biological sample collection tool has a diameter in a range from 5 to 20 mm.

12. The biological sample collection tool according to claim 10, having a spherical shape.

13. The biological sample collection tool according to claim 10, having a spindle shape.

14. The biological sample collection tool according to claim 10, having a columnar shape.

15. The biological sample collection tool according to claim 8,
wherein at least the surface of the biological sample collection tool is made of a material that includes fluororesin.

16. The biological sample collection tool according to claim 10, having the surface made of a material that includes fluororesin.
